(19) United States
(12) Patent Application Publication
(10) Pub. No.: US 2002/0058904 A1
(43) Pub. Date: May 16, 2002

(54) Thrombus removal device

(76) Inventors: Robert Boock, Minnetonka, MN (US); Kevin Gilmartin, Kingston, MA (US)

Correspondence Address: Kevin Gilmartin, 91 Ocean Hill Drive, Kingston, MA, 02364, US

(21) Appl. No.: 10/007,740

(22) Filed: Nov. 8, 2001

Related U.S. Application Data
(60) Provisional application No. 60/246,810, filed on Nov. 8, 2000.

(51) Int. Cl. A61M 1/00

(52) U.S. Cl. 604/35

(57) ABSTRACT

A method and device for clot maceration and removal, including inserting a shaft having two lumens into an occluded blood vessel, the shaft having: a guidewire/drug delivery lumen defined partially by a first wall and a thrombus removal lumen defined partially by a second wall. The thrombus removal lumen further defines a skived section where a thrombus may be trapped for removal. A portion of the first wall is coextensive with a portion of the second wall, and the coextensive portion defines at least one hole whereby a drug delivered through the guidewire/drug delivery lumen may pass to the thrombus removal lumen. A maceration device is also disclosed which may be used independently or may be inserted within the guidewire/drug delivery lumen. The maceration device includes: a base wire; a capping plug attached to a distal end of the base wire; a pressure translator slidably attached to the base wire; and a means for maceration coupled between the pressure translator and the capping plug. Variations of the above are also disclosed.

### BACKGROUND OF THE INVENTION

1. Field of the Invention

The invention relates to thrombus removal and lysis, and more particularly to devices employing removal lumens, suction, or mechanical devices to lyse or remove thrombi.

2. Description of the Related Art

Stroke afflicts more than 700,000 people in the United States yearly. Of this, 70-85% is due to ischemic stroke, and 15-30% is due to hemorrhagic stroke. Ischemic stroke carries a 15-33% mortality rate. The devastating effects of stroke and the fact that the medical community is just beginning to understand it make stroke an important topic for the neurological community.

In an acute ischemic stroke case, a patient usually enters through the emergency room and is diagnosed. Knowing the symptoms of the disease and the time frame with which physicians have to work is key to successful treatment. Quick diagnosis and treatment are the keys to limiting the effects of the disease. Every minute lost means greater impact on the patient. Providing physicians with a tool to treat ischemic stroke quickly and efficiently is vital.

The current method of treatment, intravenous drug delivery, is slow and ineffective in many cases. New stroke tools are needed that are significantly faster at reestablishing blood flow. Such tools should also incorporate current and future drug regimens to aid in maintaining proper flow and prevent further transient events (e.g. a distal protective agent). This is important for two reasons, safety and familiarity for physicians. Some physicians are slow to embrace new technology if the technology deviates too far from the familiar. In the case of ischemic stroke, there is a current need for a quick, simple method for the destruction and removal of clot, to be used alone or in conjunction with the thrombolytic treatment of stroke.

### SUMMARY OF THE INVENTION

The present invention addresses the needs discussed above.

In one aspect, the invention is directed towards a catheter for drug delivery and thrombus removal. The catheter includes a shaft having two lumens, including: a guidewire/drug delivery lumen defined partially by a first wall; and a thrombus removal lumen defined partially by a second wall, the thrombus removal lumen further defining a skived section whereby a thrombus may be trapped for removal. A portion of the first wall is coextensive with a portion of the second wall, and the coextensive portion defines at least one hole whereby a drug delivered through the guidewire/drug delivery lumen may pass to the thrombus removal lumen.

Implementations of the invention may include one or more of the following. The catheter may further include a basket disposed downstream of the skived section for containing the thrombus, and wherein the hole in the skived section is capable of delivering a drug to a volume substantially adjacent the basket. The catheter may further include a source of vacuum, wherein the source of vacuum is in pressure communication with the thrombus removal lumen via a valve. The source of vacuum may provide a vacuum at a level of between about zero and the minimum blood pressure measured at a distal end of the thrombus removal lumen. The basket may be made of polyurethane or a material of low durometer. The catheter may have an overall outer diameter of between about 0.0786 in and 0.0196 in. The guidewire/drug delivery lumen may have an overall diameter of between about 0.0343 in and 0.0085 in.

In another aspect, the invention is directed towards a method of removing a clot from a blood vessel. The method includes delivering a catheter to a blood vessel, the catheter including: a shaft having two lumens, including: a guidewire/drug delivery lumen defined partially by a first wall; and a thrombus removal lumen defined partially by a second wall, the thrombus removal lumen further defining a skived section whereby a thrombus may be trapped for removal. A portion of the first wall is coextensive with a portion of the second wall, and the coextensive portion defines at least one hole whereby a drug delivered through the guidewire/drug delivery lumen may pass to the thrombus removal lumen. After, a drug may be delivered to the clot through the guidewire/drug delivery lumen.

In another aspect, the invention is directed towards a method of removing a clot from a blood vessel. The method includes delivering a catheter to a blood vessel, the catheter including: a shaft having two lumens, including: a guidewire/drug delivery lumen defined partially by a first wall; and a thrombus removal lumen defined partially by a second wall, the thrombus removal lumen further defining a skived section whereby a thrombus may be trapped for removal. A portion of the first wall is coextensive with a portion of the second wall, and the coextensive portion defines at least one hole whereby a drug delivered through the guidewire/drug delivery lumen may pass to the thrombus removal lumen. After, the clot may be mechanically trapped in the skived section.

In another aspect, the invention is directed to a method of removing a clot from a blood vessel, including delivering a catheter to a blood vessel, the catheter including: a shaft having two lumens, including: a guidewire/drug delivery lumen defined partially by a first wall; and a thrombus removal lumen defined partially by a second wall, the thrombus removal lumen further defining a skived section whereby a thrombus may be trapped for removal. A portion of the first wall is coextensive with a portion of the second wall, and the coextensive portion defines at least one hole whereby a drug delivered through the guidewire/drug delivery lumen may pass to the thrombus removal lumen. A source of vacuum is provided and coupled in fluid communication to the thrombus removal lumen. A pressure differential is provided to the thrombus removal lumen using the vacuum source.

In another aspect, the invention is directed to a method of removing a clot from a blood vessel,including: delivering a catheter to a blood vessel, the catheter including: a shaft having two lumens, including: a guidewire/drug delivery lumen defined partially by a first wall; and a thrombus removal lumen defined partially by a second wall, the thrombus removal lumen further defining a skived section whereby a thrombus may be trapped for removal. A portion of the first wall is coextensive with a portion of the second wall, and the coextensive portion defines at least one hole whereby a drug delivered through the guidewire/drug delivery lumen may pass to the thrombus removal lumen. The catheter further includes a basket disposed downstream of the skived section for containing the thrombus. The clot may be mechanically trapped in the basket.

In another aspect, the invention is directed towards a catheter for thrombus removal, including: a shaft; a thrombus removal section distal of the shaft; and an atraumatic tip distal of the thrombus removal section, such that the thrombus removal section includes at least one scoop for removing a thrombus from a thrombosed vessel.

Implementations of the invention may include one or more of the following. The catheter may further include at least two scoops in the thrombus removal section, and the scoops may be separated by a flexible joint. The flexible joint may be selected from the group consisting of coils, polymers, and combinations thereof. The shaft, thrombus removal section, and tip may be configured and arranged to be of a size sufficiently small to pass through a standard neurological catheter. The catheter may be of a size and flexibility substantially the same as a standard neurological guidewire. The scoop is substantially made of a soft polymer.

In another aspect, the invention is directed towards a method of removing a clot, including: inserting a catheter into an occluded blood vessel. The catheter includes a shaft; a thrombus removal section distal of the shaft; and an atraumatic tip distal of the thrombus removal section. The thrombus removal section may include at least one scoop for removing a thrombus from a thrombosed vessel. A portion of a clot may be trapped in the at least one scoop.

In another aspect, the invention is directed towards a device to macerate or remove a clot. The device includes a base wire; a capping plug attached to a distal end of said base wire; a pressure translator slidably attached to the base wire; and a means for maceration coupled between the pressure translator and the capping plug.

Implementations of the invention may include one or more of the following. The maceration means may be a spring. The device may further comprise a means for providing a varying pressure on a proximal end of the pressure translator. The spring may have a diameter of between about 0.009 in and 0.038 in and a pitch of between about 0 and 45° in an unstressed state. The pressure translator may be made of a polymer having moderate stiffness. The maceration means may be selected from the group consisting of: mechanical vibrators, spring like oscillators, springs, shaped wires, microcatheters, and longitudinal filaments caused to tense and compress and expand.

In another aspect, the invention is directed towards a method of macerating a clot. The method includes disposing a device through a guide catheter. The device includes a base wire; a capping plug attached to a distal end of said base wire; a pressure translator slidably attached to the base wire; and a means for maceration coupled between the pressure translator and the capping plug. A fluid pressure is varied in a cyclical manner on the proximal end of the pressure translator.

In another aspect, the invention is directed towards a catheter for drug delivery and thrombus removal, including: a shaft having two lumens therein, including: a guidewire/drug delivery lumen defined by a guidewire/drug delivery tube; and a thrombus removal lumen defined by a suction tube; and a plurality of struts adjacent and distal from the thrombus removal tube, the plurality of struts defining a collection region, the collection region fluidically coupled to the thrombus removal lumen.

Implementations of the invention may include one or more of the following. A portion of the guidewire/drug delivery tube adjacent the collection region defines at least one hole. The catheter may further include a source of vacuum, wherein the source of vacuum is in pressure communication with the thrombus removal lumen via a valve. The source of vacuum may provide a vacuum at a level of between about zero and the minimum blood pressure at a distal end of the thrombus removal lumen. The struts may be made of a material selected from the group consisting of: polymers, stainless steel, or nitinol. The catheter has an overall outer diameter of between about 0.0786 in and 0.0196 in. The guidewire/drug delivery lumen has an overall diameter of between about 0.035 in and 0.085 in. The guidewire/drug delivery tube and the thrombus removal tube are coaxial.

In another aspect, the invention is directed to a method of removing a thrombus from a blood vessel, including: inserting a catheter into a blood vessel, the catheter including: a shaft having two lumens therein, including: a guidewire/drug delivery lumen defined by a guidewire/drug delivery tube; and a thrombus removal lumen defined by a suction tube. A plurality of struts is disposed adjacent and distal from the thrombus removal tube, the plurality of struts defining a collection region, the collection region fluidically coupled to the thrombus removal lumen. A source of suction is provided which is fluidically coupled to the proximal end of the thrombus removal lumen.

Implementations of the invention may include one or more of the following. The method may include varying the pressure provided by the source of suction in a cyclical manner.

In another aspect, the invention is directed to a catheter for clot maceration and removal, including: a shaft having two lumens therein, including: a guidewire/drug delivery lumen defined by a guidewire/drug delivery tube; and a thrombus removal lumen defined by a suction tube. A plurality of struts is disposed adjacent and distal from the thrombus removal tube, the plurality of struts defining a collection region, the collection region fluidically coupled to the thrombus removal lumen. A maceration device is insertable within the guidewire/drug delivery lumen. The maceration device includes: a base wire; a capping plug attached to a distal end of said base wire; a pressure translator slidably attached to the base wire; and a means for maceration coupled between the pressure translator and the capping plug.

In another aspect, the invention is directed to a catheter for clot maceration and removal, including: a shaft having two lumens, including: a guidewire/drug delivery lumen defined partially by a first wall; and a thrombus removal lumen defined partially by a second wall, the thrombus removal lumen further defining a skived section whereby a thrombus may be trapped for removal. A portion of the first wall is coextensive with a portion of the second wall, and the coextensive portion defines at least one hole whereby a drug delivered through the guidewire/drug delivery lumen may pass to the thrombus removal lumen. A maceration device may be insert within the guidewire/drug delivery lumen, including: a base wire; a capping plug attached to a distal end of said base wire; a pressure translator slidably attached to the base wire; and a means for maceration coupled between the pressure translator and the capping plug.

In another aspect, the invention is directed to a method of employing a catheter for clot maceration and removal. The method includes inserting a shaft having two lumens therein into an occluded blood vessel, the shaft including: a guidewire/drug delivery lumen defined by a guidewire/drug delivery tube; and a thrombus removal lumen defined by a suction tube. A plurality of struts is disposed adjacent and distal from the thrombus removal tube, the plurality of struts defining a collection region, the collection region fluidically coupled to the thrombus removal lumen. A maceration device is inserted within the guidewire/drug delivery lumen, the maceration device including: a base wire; a capping plug attached to a distal end of said base wire; a pressure translator slidably attached to the base wire; and a means for maceration coupled between the pressure translator and the capping plug.

In another aspect, the invention is directed towards a a method of employing a catheter for clot maceration and removal, comprising: inserting a shaft having two lumens into an occluded blood vessel, the shaft including: a guidewire/drug delivery lumen defined partially by a first wall; and a thrombus removal lumen defined partially by a second wall, the thrombus removal lumen further defining a skived section whereby a thrombus may be trapped for removal. A portion of the first wall is coextensive with a portion of the second wall, and the coextensive portion defines at least one hole whereby a drug delivered through the guidewire/drug delivery lumen may pass to the thrombus removal lumen. A maceration device is inserted within the guidewire/drug delivery lumen. The maceration device includes: a base wire; a capping plug attached to a distal end of said base wire; a pressure translator slidably attached to the base wire; and a means for maceration coupled between the pressure translator and the capping plug.

Advantages of the invention include one or more of the following. The device is disposable and combines mechanical clot disruption, removal, and the ability to deliver drug directly into or distally to the ischemic event. The device is a combination of a catheter, which is capable of removing clot and delivering drugs, and a small over-the-wire device capable of breaking up clot. The device is advantageous as it is able to simultaneously deliver drug, mechanically disrupt, and remove the clot. The device supplies the physician with a clinically superior tool with which to treat stroke.

The device gives physicians (e.g., interventional neuro radiologists) with a tool to immediately open the infarcted area and prevent further damage of brain tissue. The device and method can be done in concert with or without delivering drugs. The device combines a multilumen microcatheter with a guidewire-like device designed to break up clot.

The device can be used to remove, e.g., soft clot (young embolus or clot with some calcified particles). When use with a drug therapy, the device lessens the chance of spreading the ischemic event distally in the brain. The device also provides the ability to deliver neuro protective agents prior to reestablishing flow. Such an ability helps to prevent reprofusion injury once flow has been reestablished. Combining the mechanical removal and effects of clot busting drugs may significantly reduce the time required to reestablish flow, thereby limiting the amount of damage caused by the lack of oxygen to the brain tissue. The device is simple to use and provides the physician with quick and easy access to the affected site.

### BRIEF DESCRIPTION OF THE DRAWINGS

FIG. 1A is a schematic cutaway view of a first embodiment of a thrombus removal device.

FIG. 1B is a schematic view of a thrombosed vessel, which may advantageously employ the device of FIG. 1A.

FIG. 1C is a schematic view of the device of FIG. 1A in use within the thrombosed vessel shown in FIG. 1B.

FIG. 2A is a schematic cutaway view of a second embodiment of a thrombus removal device including a thrombus removal basket.

FIG. 2B is a schematic view of a thrombosed vessel, which may advantageously employ the device of FIG. 2A.

FIG. 2C is a schematic view of the device of FIG. 2A in use within the thrombosed vessel shown in FIG. 2B.

FIG. 3A is a schematic view of a third embodiment of a thrombus removal device including a thrombus macerator, the latter in an extended position. In this specification, the terms macerator, thrombus macerator, and clot disruption device are used interchangeably.

FIG. 3B is a schematic view of a third embodiment of a thrombus removal device including a thrombus macerator, the latter in a contracted position.

FIG. 4 is a schematic view of a fourth embodiment of a thrombus removal device.

FIG. 5A is a schematic view of a fifth embodiment of a thrombus removal device.

FIG. 5B is a schematic cross-sectional view of the thrombus removal device of FIG. 5A.

### DETAILED DESCRIPTION OF THE PREFERRED EMBODIMENTS

### First Embodiment

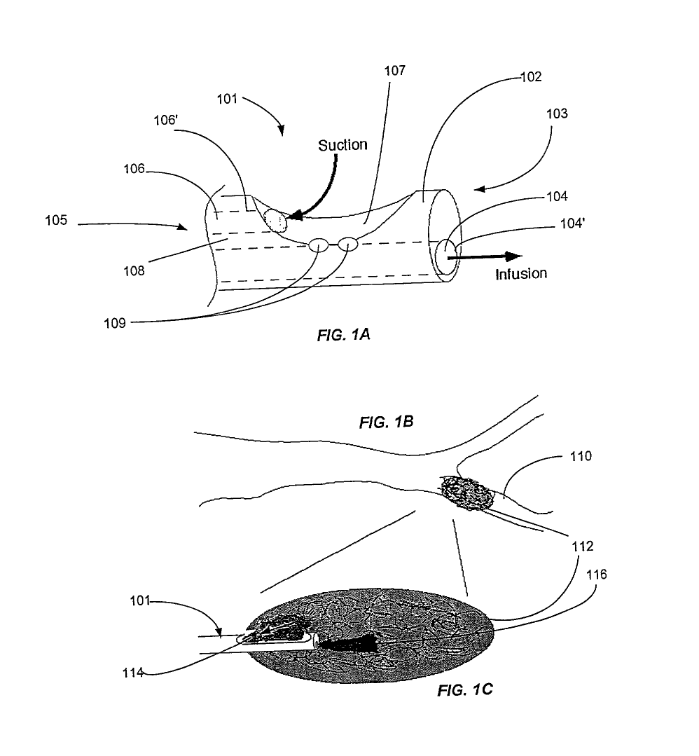

Referring to FIG. 1A, a first embodiment of a thrombus removal device 101 is shown. The overall outside diameter of such a device may be, e.g., about 3 French although this will vary widely with the application. The thrombus removal device 101 has a distal end 103 and a proximal end 105. The proximal end 105 shown is schematic in nature and is not shown to scale. The proximal end 105 may more generally be located at the opposite end of a catheter embodying the thrombus removal device 101. Externally introduced fluids, liquids and gases may be provided to the thrombus removal device 101 at its proximal end 105 for distribution to a thrombosed area or volume at the distal end 103. Conversely, a suction or source of vacuum may be applied to the proximal end 105 in order to cause biological agents, such as clots, to move in a proximal direction relative to the distal end 103.

A distal tip 102 is located adjacent the distal end 103. The distal tip 102 forms the terminal end of a thrombus removal lumen 106. The thrombus removal lumen 106 has, adjacent the distal tip 102, a skived section 107. The skived section 107 may be formed by an automatic tool or by hand with a sharp blade. The skived section 107 is used to remove thrombus in a manner explained below.

A guidewire/drug delivery lumen 104 is defined by a first wall 104′. In a similar manner, the thrombus removal lumen 106 is defined by a second wall 106′. The first wall 104′ and the second wall 106′ are coextensive along a wall portion 108. The guidewire/drug delivery lumen 104 is generally of a throughlumen design, i.e., the same passes throughout the length of the catheter. The guidewire/drug delivery lumen 104 may include a lubricious inner liner to improve guidewire movement and may be employed in a manner explained below.

Referring to FIG. 1B, a thrombosed vessel 110 is shown with a thrombus 112 located therein. The thrombus 112 generally causes a blood flow obstruction that may be minor or total. In FIG. 1B, the thrombus 112 shows a total obstruction.

A guidewire may initially be placed in a known manner with a distal end of the guidewire disposed at, proximal to, or distal to a thrombus. In cases where the thrombus causes a near or total obstruction, the guidewire may be placed proximal to the thrombus or may alternatively pierce or cannulate the thrombus. Suitable sizes for such guidewires include 0.014 in (in which case the guidewire/drug delivery lumen 104 may be 0.017 in.). As another example, the guidewire may be 0.010 in (in which case the guidewire/drug delivery lumen 104 may be 0.013 in to 0.014 in.).

Once guidewire placement is completed, the thrombus removal device 101 may be threaded onto the guidewire along the guidewire/drug delivery lumen 104 and maneuvered to the location of the thrombus. The device 101 may be partially or totally embedded in the thrombus 112 as is shown in FIG. 1C. Radioopaque markers may be used to identify accurately the position of the skive. The guidewire/drug delivery lumen 104 may be employed to pass drugs such as thrombolytics to the location of the clot.

The thrombus removal lumen 106 may be attached to a source of suction or vacuum to mechanically force, via a pressure gradient, clots or thrombi from a distal end of the thrombus removal lumen 106 to a proximal end of the thrombus removal lumen 106. A suitable pressure for such a gradient may be about just above zero to the minimum blood pressure at the point of occlusion. Of course, as may be seen from FIG. 1C, the diameter of the thrombus removal lumen 106 may be significantly smaller than the clot or thrombus 112 being treated. In this case, pieces or portions of the thrombus may be removed individually or collectively as is indicated generally by a clot portion 114. Even with only a partial removal, a significant portion of the blood vessel may be cleared for blood flow. During this time, lytics may be passed into the clot as indicated generally be a lytic portion 116.

A number of pass-through holes 109 (two are shown) may be provided between the guidewire/drug delivery lumen 104 and the thrombus removal lumen 106 and, in particular, the skived section 107 to assist in dissolving the clot using thrombolytic drugs. These drugs may also help prevent clogging of the thrombus removal lumen 106.

Of course, it is clear that clot removal via suction is not necessary for successful practice of the device. For example, the suction may be avoided completely. Alternatively, the suction may be used to attach a clot to the device while the entire device is withdrawn from the vessel. Also alternatively, the device may provide for initial obstruction cannulation while the same is still on the guidewire.

### Second Embodiment

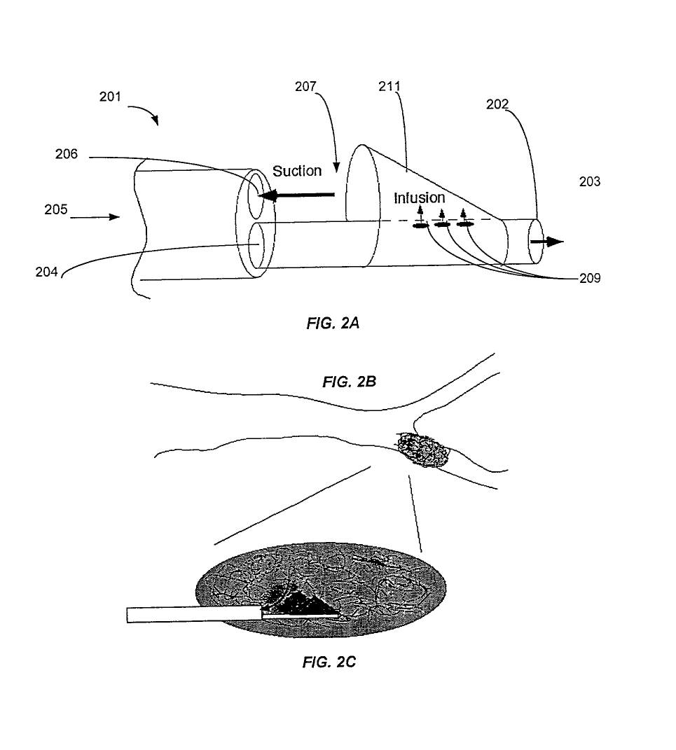

Referring to FIG. 2A, a second embodiment of a thrombus removal device 201 is shown having various features similar to those of FIG. 1A. The thrombus removal device 201 has a distal end 203 and a proximal end 205. A suction or source of vacuum may be applied to the proximal end 205 in order to cause biological agents to move proximal to the distal end 203. A distal tip 202 is located adjacent the distal end 203. The distal tip 202 forms the terminal end of a thrombus removal lumen 206. The thrombus removal lumen 206 may have a cylindrical section 207. The cylindrical section 207 generally corresponds to skived section 107.

A guidewire/drug delivery lumen 204 is defined by a first wall similar to first wall 104′. In a similar manner, the thrombus removal lumen 206 is defined by a second wall similar to second wall 106′. The first wall and the second wall are coextensive along a wall portion. The guidewire/drug delivery lumen 204 is generally of a throughlumen design, i.e., the same passes throughout the length of the catheter.

Referring to FIG. 2B, a thrombosed vessel is shown with a thrombus located therein. The action of device 201 may be generally similar to that of device 101 above with respect to drug delivery and clot suction. Furthermore, the device 201 may be placed in a desired location similarly using a guidewire having the exemplary dimensions above.

A number of pass-through or “weep” holes 209 may be provided to pass drugs from the guidewire/drug delivery lumen 204 to the skived section 207 to assist in dissolving the clot using thrombolytic drugs.

In this second embodiment, a thrombus removal basket 211 is provided for mechanical thrombus removal. The basket 211 is of a generally soft material to prevent trauma to blood vessel walls. However, the basket 211 should be made of a material that is stiff enough to maintain its shape. Such materials include soft polymers such as polyurethane, or alternatively equivalent materials of low durometer. In an alternative embodiment, a Nitinol wire may be wrapped in a “hoop” fashion around the basket 211 to assist in maintaining its shape.

In use, the basket 211 is disposed during installation such that the clot is between the basket 211 and the thrombus removal lumen 206, e.g., adjacent the cylindrical section 207. The clot may be caught in the basket 211. By removing the entire device 201 from the blood vessel, the clot can thus be removed from the blood vessel.

### Third Embodiment

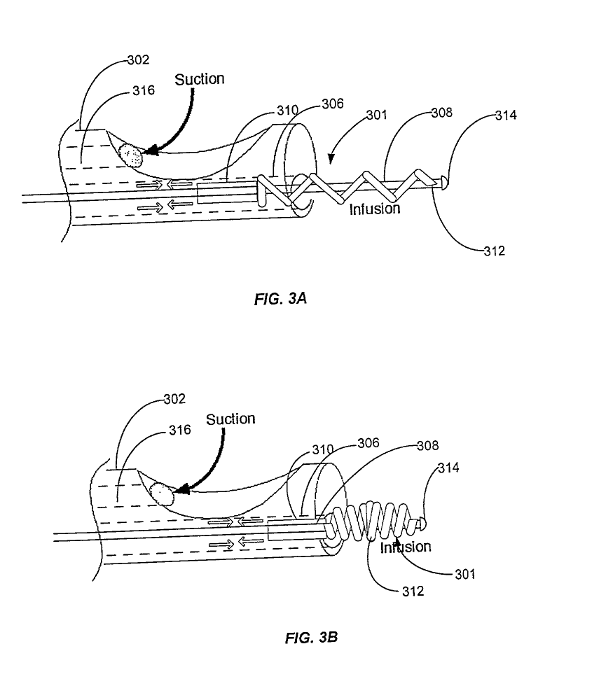

In a third embodiment of the invention, referring in particular to FIGS. 3A and 3B, a device is shown which may be especially useful for removing soft clots, although to a certain extent older aged clots and calcified lesions may also be removed. FIG. 3A shows the device of the third embodiment in an extended or relaxed position, and FIG. 3B shows the same device in a contracted position, as will be explained.

Referring to FIG. 3A, a catheter 302 is shown having a vacuum lumen 316 and an auxiliary lumen 306 which may be alternatively used for, e.g., drug delivery, guidewire placement, or microcatheter delivery. As may be apparent, the catheter 302 can have the same form as the catheter 102 of FIG. 1A. In fact, a microcatheter 301 shown in FIGS. 3A and 3B may be employed through the infusion lumen 104 of the catheter 102 of FIG. 1A or may alternatively be employed in the catheter of FIGS. 2A and 2B.

The microcatheter 301 includes a base wire 308, a pressure translator 310, a means for maceration such as spring 312, and a capping plug 314. The base wire 308 generally extends the length of the microcatheter 301 and is used to translate a varying pressure differential at the proximal end of the microcatheter to the distal end of the microcatheter. The base wire 308 may have a diameter of from about 0.001 in to 0.014 in and may be made of stainless steel or nitinol or other similar materials.

The pressure translator 310 is, in its simplest form, a block slidably attached to the base wire 308. The pressure translator 310 generally has a diameter just less than the auxiliary lumen 306. The pressure translator 310 may have a diameter of from about 0.014 to 0.016 in and may be made of a semi-rigid polymer to translate force. As the pressure translator 310 is intended to slide easily through auxiliary lumen 306, either or both may be provided with a lubricious coating such as a hydrophilic coating or TFE (Teflon®) or other similar coating. Upstream of the pressure translator 310 is provided a source of varying pressure, such as a pressure-varying source of saline. The action of the source of varying pressure is indicated in FIGS. 3A and 3B by arrows. As the pressure translator 310 is slidably attached to base wire 308, the source of varying pressure forces the pressure translator 310 to oscillate back and forth in response to the pressure, even though the base wire 308 and the capping plug 314 are substantially stationery.

A means for maceration is then provided between the pressure translator 310 and the capping plug 314. The means for maceration may be a spring 312 as is indicated in FIGS. 3A and 3B. Alternatively, the means for maceration may be a guidewire or a wire with a specific geometric shape, e.g., one similar to a rasp. The spring 312 may be fixed at its distal end, roughly adjacent the capping plug 314, but its proximal end may be attached to the slidably attached pressure translator 310. In this way, the spring is forced to expand (FIG. 3A) and contract (FIG. 3B) at the same rate and at the same time as the pressure translator 310 is forced to oscillate. The action of the spring in expanding and contracting is to alter or push against the mechanical environment of the blood or tissue in its immediate vicinity. Accordingly, if the means for maceration is disposed in a blood clot, forcing the pressure translator 310 to oscillate forces the spring to mechanically alter the clot, which can cause destruction of the clot. For example, some springs will alter diameter when expanded and contracted. Others will keep the same diameter but may macerate by cutting clot cells which may then get trapped between turns of the spring. Numerous mechanisms may occur but the end effect is that the clot is destroyed. It should be noted that the spring 312 is effective whether or not the design of the spring is such that the diameter varies with respect to the degree of contraction. Once the clot is macerated or destroyed, the vacuum lumen 316 may be used to remove the macerated or destroyed clot cells from the blood vessel.

### Fourth Embodiment

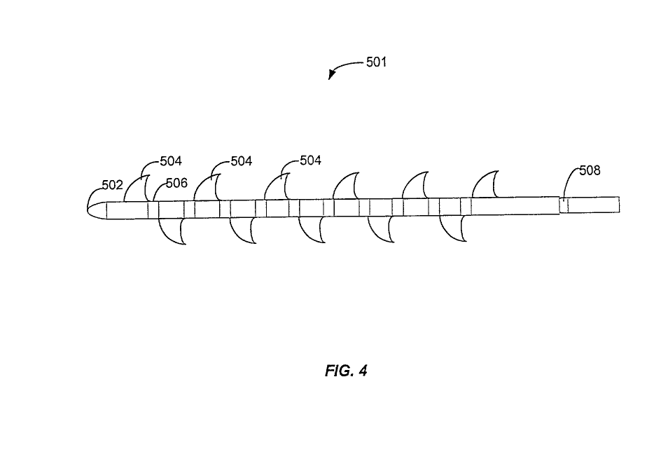

A fourth embodiment of the invention is shown in FIG. 4. In this figure, a clot removal guidewire 501 is shown with at least one clot removal scoop 504 mounted thereon. The clot removal scoops 504 may be made of soft polymers or other similar materials. Between scoops 504 are disposed flexible joints 506. The flexible joints 506 may be coils, polymers, or may be made of other similar materials. A soft atraumatic tip may be disposed at the distal end of the clot removal guidewire 501. The tip may be made of a polymer or other soft material, including soft coils. The overall outer diameter of the clot removal guidewire 501 may be similar to that of current guidewires, such as about 0.009 in to 0.038 in, although this diameter may vary.

In use, the guidewire 501 may be passed through the clot or adjacent to the clot. This may be assisted by the small diameter of the guidewire 501. Before entering and once through the clot, the scoops 504 expand into a basket shape as shown in the figure due to their inherent material stiffness. However, while passing through the clot in a distal direction (to the left in FIG. 4), the baskets may attain a streamlined shape due to their inherent flexibility. When the guidewire 501 is withdrawn by the operator in a proximal direction (to the right in FIG. 4), the scoops 504 capture small portions of the clot and allow the same to be removed from the bloodstream.

An optional feature may be to use a tapered guidewire shaft 508 connected to the proximal end of the guidewire 501. The tapered guidewire shaft 508 may provide the majority of the overall catheter, with the portion of the guidewire 501 containing scoops 504 merely providing the distal tip. In this way, a highly flexible catheter may be provided in order to navigate tortuous vasculature.

### Fifth Embodiment

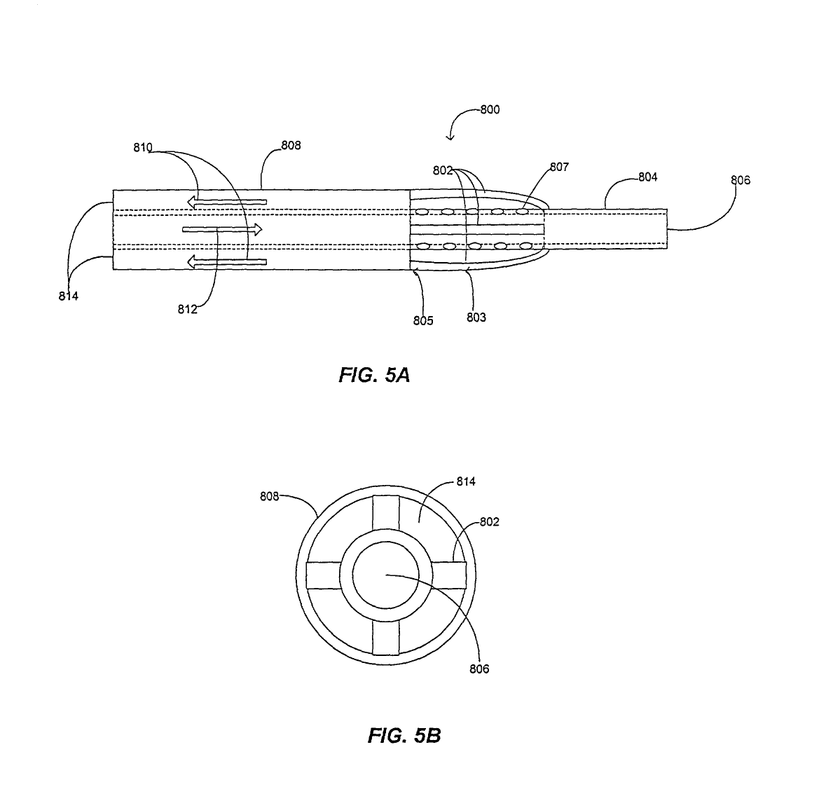

A fifth embodiment of a thrombus removal device is shown in FIGS. 5A and 5B. In particular, a thrombus removal device 800 is shown including a catheter 808. Running inside the catheter 808 is a guidewire/drug delivery tube 804 which defines a guidewire/drug delivery lumen 806. Between the guidewire/drug delivery tube 804 and the interior wall of catheter 808 is a suction lumen 814, the suction lumen 814 also referred to as a thrombus removal lumen 814. A plurality of struts 802 define a collection region 803. This collection region may have the general shape of a basket, for example. FIG. 5A shows that the suction lumen 814 need only be present between a distal point 815 and the proximal end (not shown) of the catheter; thus, the distal point 815 is also the distal end of the suction lumen 814.

Suitable materials for the catheter 808 include polyurethane, pebax, polyethelene, polypropelene, nylon, as well as other similar materials. Suitable materials for the struts 802 include stainless steel, liquid crystal polymer material, polyurethane, polypropelene, and nylon, as well as other similar materials. An appropriate dimension for the struts 802 may be about 0.003″ wide by 0.001″ thick by 5 to 10 cm long, with appropriate variations, in order to capture a large proportion of clots.

In use, the catheter 800 may be threaded onto a guidewire (not shown), using guidewire/drug delivery lumen 806, and disposed at a location such that the collection region 803 is adjacent a clot or other such thrombus or obstruction. A source of suction may be fluidically coupled to the suction lumen 814. If the clot is small, the same may enter the collection region 803 and be removed via the suction from the suction lumen 814. If the clot is large, the same may be broken up by first being attracted to the collection region 803 via the action of the suction and second by being mechanically broken up via impact against the struts 802.

Alternatively, a series of “weep” holes 807 may be provided in the guidewire/drug delivery tube 804 in order to communicate a thrombolytic drug to the collection region 803 adjacent the clot or obstruction. Alternatively, such a drug may be delivered via the distal end of the catheter using the guidewire/drug delivery lumen 806 prior to or contemporaneous with the placement of the catheter. Appropriate drugs for such purposes include r-tPA, TNK-TPA, activase, urokinase, pro-urokinase, streptokinase, alteplase, tenactoplase, retoplase, abicxomib, and Rheopro.

Although specific embodiments of the invention have been described above, it is clear to one of skill in the art that variations of the above may be employed which still fall within the spirit of the invention. For example, a basket made of longitudinal and radial supports, laser or photo etched net like pattern or a plate or tube of various geometry.

Accordingly, the invention is limited only the scope of the claims appended hereto, and equivalents thereof.

What is claimed is:

1. A catheter for drug delivery and thrombus removal, comprising:
a shaft having two lumens, including:
a guidewire/drug delivery lumen defined partially by a first wall; and
a thrombus removal lumen defined partially by a second wall, the thrombus removal lumen further defining a skived section whereby a thrombus may be trapped for removal,
such that a portion of the first wall is coextensive with a portion of the second wall, and the coextensive portion defines at least one hole whereby a drug delivered through the guidewire/drug delivery lumen may pass to the thrombus removal lumen.

2. The catheter of claim 1, wherein the catheter further includes a basket disposed downstream of the skived section for containing the thrombus, and wherein the hole in the skived section is capable of delivering a drug to a volume substantially adjacent the basket.

3. The catheter of claim 1, further comprising a source of vacuum, wherein the source of vacuum is in pressure communication with the thrombus removal lumen via a valve.

4. The catheter of claim 3, wherein the source of vacuum provides a vacuum at a level of between about zero and the minimum blood pressure measured at a distal end of the thrombus removal lumen.

5. The catheter of claim 2, wherein the basket is made of polyurethane or a material of low durometer.

6. The catheter of claim 1, wherein the catheter has an overall outer diameter of between about 0.0786 in and 0.0196 in.

7. The catheter of claim 1, wherein the guidewire/drug delivery lumen has an overall diameter of between about 0.0343 in and 0.0085 in.

8. A method of removing a clot from a blood vessel, comprising:
delivering a catheter to a blood vessel, the catheter including:
a shaft having two lumens, including:
a guidewire/drug delivery lumen defined partially by a first wall; and
a thrombus removal lumen defined partially by a second wall, the thrombus removal lumen further defining a skived section whereby a thrombus may be trapped for removal,
such that a portion of the first wall is coextensive with a portion of the second wall, and the coextensive portion defines at least one hole whereby a drug delivered through the guidewire/drug delivery lumen may pass to the thrombus removal lumen; and
delivering a drug to the clot through the guidewire/drug delivery lumen.

9. A method of removing a clot from a blood vessel, comprising:
delivering a catheter to a blood vessel, the catheter including:
a shaft having two lumens, including:
a guidewire/drug delivery lumen defined partially by a first wall; and
a thrombus removal lumen defined partially by a second wall, the thrombus removal lumen further defining a skived section whereby a thrombus may be trapped for removal,
such that a portion of the first wall is coextensive with a portion of the second wall, and the coextensive portion defines at least one hole whereby a drug delivered through the guidewire/drug delivery lumen may pass to the thrombus removal lumen; and
mechanically trapping the clot in the skived section.

10. A method of removing a clot from a blood vessel, comprising:
delivering a catheter to a blood vessel, the catheter including:
a shaft having two lumens, including:
a guidewire/drug delivery lumen defined partially by a first wall; and
a thrombus removal lumen defined partially by a second wall, the thrombus removal lumen further defining a skived section whereby a thrombus may be trapped for removal,
such that a portion of the first wall is coextensive with a portion of the second wall, and the coextensive portion defines at least one hole whereby a drug delivered through the guidewire/drug delivery lumen may pass to the thrombus removal lumen; and
providing a source of vacuum coupled in fluid communication to the thrombus removal lumen;
providing a pressure differential to the thrombus removal lumen using the vacuum source.

11. A method of removing a clot from a blood vessel, comprising:
delivering a catheter to a blood vessel, the catheter including:
a shaft having two lumens, including:
a guidewire/drug delivery lumen defined partially by a first wall; and
a thrombus removal lumen defined partially by a second wall, the thrombus removal lumen further defining a skived section whereby a thrombus may be trapped for removal,
such that a portion of the first wall is coextensive with a portion of the second wall, and the coextensive portion defines at least one hole whereby a drug delivered through the guidewire/drug delivery lumen may pass to the thrombus removal lumen, and
wherein the catheter further includes a basket disposed downstream of the skived section for containing the thrombus;
mechanically trapping the clot in the basket.

12. A catheter for thrombus removal, comprising:
a shaft;
a thrombus removal section distal of the shaft; and
an atraumatic tip distal of the thrombus removal section,
such that the thrombus removal section includes at least one scoop for removing a thrombus from a thrombosed vessel.

13. The catheter of claim 12, further comprising at least two scoops in the thrombus removal section, and wherein the scoops are separated by a flexible joint.

14. The catheter of claim 13, wherein the flexible joint is selected from the group consisting of coils, polymers, and combinations thereof.

15. The catheter of claim 12, wherein the shaft, thrombus removal section, and tip are configured and arranged to be of a size sufficiently small to pass through a standard neurological catheter.

16. The catheter of claim 12, wherein the catheter is of a size and flexibility substantially the same as a standard neurological guidewire.

17. The catheter of claim 12, wherein the scoop is substantially made of a soft polymer.

18. A method of removing a clot, comprising:
inserting a catheter into an occluded blood vessel, the catheter including:
a shaft;
a thrombus removal section distal of the shaft; and
an atraumatic tip distal of the thrombus removal section,
such that the thrombus removal section includes at least one scoop for removing a thrombus from a thrombosed vessel; and
trapping a portion of a clot in the at least one scoop.

19. A device to macerate or remove a clot, comprising:
a base wire;
a capping plug attached to a distal end of said base wire;
a pressure translator slidably attached to the base wire; and
a means for maceration coupled between the pressure translator and the capping plug.

20. The device of claim 19, wherein the maceration means is a spring.

21. The device of claim 19, further comprising a means for providing a varying pressure on a proximal end of the pressure translator.

22. The device of claim 19, wherein the spring has a diameter of between about 0.009 in and 0.038 in and a pitch of between about 0 and 45° in an unstressed state.

23. The device of claim 19, wherein the pressure translator is made of a polymer having moderate stiffness.

24. The device of claim 19, wherein the maceration means is selected from the group consisting of: mechanical vibrators, spring like oscillators, springs, shaped wires, microcatheters, and longitudinal filaments caused to tense and compress and expand.

25. A method of macerating a clot, comprising:
disposing a device through a guide catheter, the device including:
a base wire;
a capping plug attached to a distal end of said base wire;
a pressure translator slidably attached to the base wire; and
a means for maceration coupled between the pressure translator and the capping plug; and
varying a fluid pressure in a cyclical manner on the proximal end of the pressure translator.

26. A catheter for drug delivery and thrombus removal, comprising:
a shaft having two lumens therein, including:
a guidewire/drug delivery lumen defined by a guidewire/drug delivery tube; and
a thrombus removal lumen defined by a suction tube;
a plurality of struts adjacent and distal from the thrombus removal tube, the plurality of struts defining a collection region, the collection region fluidically coupled to the thrombus removal lumen.

27. The catheter of claim 26, wherein a portion of the guidewire/drug delivery tube adjacent the collection region defines at least one hole.

28. The catheter of claim 26, further comprising a source of vacuum, wherein a source of vacuum is in pressure communication with the thrombus removal lumen via a valve.

29. The catheter of claim 28, wherein the source of vacuum provides a vacuum at a level of between about zero and the minimum blood pressure at a distal end of the thrombus removal lumen.

30. The catheter of claim 26, wherein the struts are made of a material selected from the group consisting of: polymers, stainless steel, or nitinol.

31. The catheter of claim 26, wherein the catheter has an overall outer diameter of between about 0.0786 in and 0.0196 in.

32. The catheter of claim 26, wherein the guidewire/drug delivery lumen has an overall diameter of between about 0.035 in and 0.085 in.

33. The catheter of claim 26, wherein the guidewire/drug delivery tube and the thrombus removal tube are coaxial.

34. A method of removing a thrombus from a blood vessel, comprising:
inserting a catheter into a blood vessel, the catheter including:
a shaft having two lumens therein, including:
a guidewire/drug delivery lumen defined by a guidewire/drug delivery tube; and
a thrombus removal lumen defined by a suction tube;
a plurality of struts adjacent and distal from the thrombus removal tube, the plurality of struts defining a collection region, the collection region fluidically coupled to the thrombus removal lumen; and
providing a source of suction fluidically coupled to the proximal end of the thrombus removal lumen.

35. The method of claim 34, further comprising varying the pressure provided by the source of suction in a cyclical manner.

36. A catheter for clot maceration and removal, comprising:
a shaft having two lumens therein, including:
a guidewire/drug delivery lumen defined by a guidewire/drug delivery tube; and
a thrombus removal lumen defined by a suction tube;
a plurality of struts adjacent and distal from the thrombus removal tube, the plurality of struts defining a collection region, the collection region fluidically coupled to the thrombus removal lumen; and
a maceration device to insert within the guidewire/drug delivery lumen, including:
a base wire;
a capping plug attached to a distal end of said base wire;
a pressure translator slidably attached to the base wire; and
a means for maceration coupled between the pressure translator and the capping plug.

37. A catheter for clot maceration and removal, comprising:
a shaft having two lumens, including:
a guidewire/drug delivery lumen defined partially by a first wall; and
a thrombus removal lumen defined partially by a second wall, the thrombus removal lumen further defining a skived section whereby a thrombus may be trapped for removal,
such that a portion of the first wall is coextensive with a portion of the second wall, and the coextensive portion defines at least one hole whereby a drug delivered through the guidewire/drug delivery lumen may pass to the thrombus removal lumen; and
a maceration device to insert within the guidewire/drug delivery lumen, including:
a base wire;
a capping plug attached to a distal end of said base wire;
a pressure translator slidably attached to the base wire; and
a means for maceration coupled between the pressure translator and the capping plug.

38. A method of employing a catheter for clot maceration and removal, comprising:
inserting a shaft having two lumens therein into an occluded blood vessel, the shaft including:
a guidewire/drug delivery lumen defined by a guidewire/drug delivery tube; and
a thrombus removal lumen defined by a suction tube;
a plurality of struts adjacent and distal from the thrombus removal tube, the plurality of struts defining a collection region, the collection region fluidically coupled to the thrombus removal lumen; and
inserting a maceration device within the guidewire/drug delivery lumen, the maceration device including:
a base wire;
a capping plug attached to a distal end of said base wire;
a pressure translator slidably attached to the base wire; and
a means for maceration coupled between the pressure translator and the capping plug.

39. A method of employing a catheter for clot maceration and removal, comprising:
inserting a shaft having two lumens into an occluded blood vessel, the shaft including:
a guidewire/drug delivery lumen defined partially by a first wall; and
a thrombus removal lumen defined partially by a second wall, the thrombus removal lumen further defining a skived section whereby a thrombus may be trapped for removal,
such that a portion of the first wall is coextensive with a portion of the second wall, and the coextensive portion defines at least one hole whereby a drug delivered through the guidewire/drug delivery lumen may pass to the thrombus removal lumen; and
inserting a maceration device within the guidewire/drug delivery lumen, the maceration device including:
a base wire;
a capping plug attached to a distal end of said base wire;
a pressure translator slidably attached to the base wire; and
a means for maceration coupled between the pressure translator and the capping plug.
